(19) United States
(12) Patent Application Publication
(10) Pub. No.: US 2018/0224378 A1
(43) Pub. Date: Aug. 9, 2018

(54) OPTICAL DETECTION UNIT

(71) Applicants: Molecular Vision Limited, York (GB); Cambridge Display Technology Limited, Cambridgeshire (GB)

(72) Inventors: Christopher John Kay, Cambridgeshire (GB); Chris Hand, York (GB); Oliver Hofmann, Greater London (GB); Gihan Ryu, Surrey (GB); Miguel Ramon, Cambridgeshire (GB); llaria Grizzi, Cambridgeshire (GB)

(73) Assignees: Molecular Vision Limited, York (GB); Cambridge Display Technology Limited, Cambridgeshire (GB)

(21) Appl. No.: 15/751,060

(22) PCT Filed: Aug. 19, 2016
(86) PCT No.: PCT/GB2016/052587

§ 371 Date: Feb. 7, 2018

(30) Foreign Application Priority Data
Aug. 19, 2015 (GB) 1514735.8

(51) Int. Cl. G01N 21/77; B01L 3/00; G01N 33/558; G01N 33/543; H01L 51/00; G01N 33/58; H01L 27/28

(52) U.S. Cl. CPC G01N 21/77; B01L 3/5023; G01N 33/558; G01N 33/54386; H01L 51/0085; H01L 51/0046; G01N 2021/7783; G01N 33/582; H01L 27/288; B01L 2300/0645; B01L 2400/0406; B01L 2300/12; B01L 2300/069; H01L 51/0036

(57) ABSTRACT

An improved optical detection unit for an assay device, such as a lateral flow device, for the quantitative determination of the concentration of an analyte in a liquid sample, and an assay device comprising the same. The detection unit comprises an organic light emitting diode (OLED) emitter that has an emission spectrum E within the wavelength range from λ1 to λ2, and an organic photodiode detector (OPD) that has a light detection spectrum S within the wavelength range from λ1 to λ2. The detection unit has a test region that comprises a light absorbing component that has an absorbance spectrum A within the wavelength range from λ1 to λ2. The test region is positioned adjacent to the emitter and the detector to form an optical pathway from the light emitting diode to the photodiode through at least a portion of the test region. Formula M defines a relationship between E, S and A, and M is less than about 0.4.

The present invention relates to an improved optical detection unit for an assay device for the quantitative determination of the concentration of an analyte in a liquid sample and to an assay device comprising the detection unit. The light emitter of the unit may be an organic light emitting diode (OLED), the light detector may be an organic photodetector (OPD), and the sample may be a liquid biological sample, e.g. plasma, serum, saliva or urine, or a biological sample reduced to a liquid.

### BACKGROUND

Lateral flow devices (LFDs) are one example of an assay device that may use optical means for detecting an analyte in a liquid sample, and have found considerable use. One of the applications is in devices that analyse a liquid sample to determine the presence or absence of one or more target analytes that may be in the sample. In these devices there is usually a threshold concentration which, when exceeded, results in an qualitative indication that a target analyte is present.

Several techniques have been developed for producing a quantitative measurement of the concentration of a target analyte, for example using light receptors coupled with a light source. Within this field, there are two broad sub-classes. One uses the detection of the reflected emission from the light source. In this class, both the light source and the light detector are provided on the same side of, for example, a lateral flow membrane. An alternative class positions the light source and the light detector on opposite sides of the lateral flow membrane such that the light (or other electromagnetic radiation) is transmitted through the membrane to the detector.

WO 2005/111579 is a transmission-based luminescent detection system.

The light source and the light detector may comprise inorganic optoelectronic components. For example, inorganic LEDs provide a bright point source that may be combined with diffusers, lenses, or other light shaping components to provide the uniform irradiation of a sample required for absorbance measurements. Inorganic photodetectors suitable for use as detectors in these applications may include silicon photodiodes, phototransistors or photoresistors. Other components such as diffusers, lenses or optical filters may also be required in order to accurately measure the required absorbance change in a sample.

Organic light sources and detectors provide certain advantages not readily obtainable from their inorganic counterparts, and are increasingly used. For example, organic light emitting diodes (OLEDs) can provide diffuse area irradiation of a sample without the need for additional components, and their typically planar construction is well adapted for irradiation of liquid samples in a flow cell, cuvette, or lateral flow device, as it permits close approach between the sample and light source. Similarly, the light detector can be an organic photodiode (OPD), which can also provide diffuse area detection without the need for additional components, and likewise can advantageously be positioned close to the sample.

A significant further benefit of organic light sources and light detectors compared to their inorganic counterparts is the degree to which their absorption or emission spectra may be adapted to a particular application. For both OLEDs and OPDs, a wide range of organic photoactive materials with different absorption or emission spectra are known. Further, features of the device structure can be adapted in order to tune the absorption and emission spectra. For example, varying the thickness of active layers of OLEDs and OPDs, the composition of adjacent non-emissive or non-absorptive players such as charge transporting layers, or outcoupling structures such as Bragg filters or microcavities, can all affect the absorption or emission spectra of organic devices.

Where the devices is a lateral flow device, a variety of light quenching detection labels are available. Typically these labels are attached to antibodies that, during the course of the assay, become immobilised within the light path of the optical emitter and detector. The label may be a small organic molecule with a high extinction coefficient in which case absorption measurements can be used to determine the amount of the label, or the label may be a light scattering particle such as a latex particle, or the label may be a metal particle such as a gold nanoparticle with complex optical properties. All of these optical changes are herein grouped within the term “absorption”.

A designer of an assay device for the quantitative determination of an analyte is therefore presented with a complex set of decisions in selecting the optimum available emitter, detector, and absorbing label for a particular application, and in quantifying the benefits or disadvantages of subtle structural changes in the emitter or detector. Optimising these choices is necessary to achieve the best performance from such assay devices.

There is therefore a need in the art for an assay device comprising an improved and preferably optimised optical detection unit for an assay device for the quantitative determination of the concentration of an analyte in a liquid sample.

The present invention, at least in its preferred embodiments, aims to provide such improved or optimized assay devices.

### BRIEF SUMMARY OF THE DISCLOSURE

In accordance with the present disclosure there is provided an optical detection unit for an assay device for the quantitative determination of the concentration of an analyte in a liquid sample. The detection unit comprises an organic light emitting diode (OLED) emitter that has an emission spectrum E within the wavelength range from λ₁ to λ₂, and an organic photodiode detector (OPD) that has a light detection spectrum S within the wavelength range from λ₁ to λ₂. The detection unit has a test region that comprises a light absorbing component that has an absorbance spectrum A within the wavelength range from λ₁ to λ₂. The test region is positioned adjacent to the emitter and the detector to form an optical pathway from the light emitting diode to the photodiode through at least a portion of the test region. Formula M defines a relationship between E, S and A and M is less than about 0.4:

$$M = \frac{{\int }_{λ1}^{λ2} {10}^{- A} . E . S d λ}{{\int }_{λ1}^{λ2} E . S d λ} .$$

Thus, in accordance with the disclosure, the optical detection unit provides a relatively simple construction that comprises an organic emitter, organic detector, and light absorbing component that have spectra that are matched to each other in a manner that provides improved detection of the absorbing component. The present inventors have found that optical detection is surprisingly improved in organic absorbance detection units when the spectra of organic emitter, organic detector, and light absorbing component are matched according to the above equation and the unit is constructed to have an M value of less than about 0.4. Preferably, the value of M is less than about 0.3, more preferably the value of M is less than about 0.2, and most preferably the value of M is less than about 0.1. These values of M permit absorbance measurements to be made using the unit with low background signal, reduced noise, and thereby enable more sensitive and accurate absorbance measurements.

In organic devices the extent to which the spectra of the organic emitter and organic detector may be adjusted by the selection of active materials and device construction is considerable. Thus in certain embodiments the required M value can be met without the need for an optical filter such as a narrow bandpass filter that may otherwise be required for example if using inorganic emitters or detectors.

In certain embodiments, the test region may be a light transmissive lateral flow membrane such as, for example, a nitrocellulose membrane as may be used in a lateral flow device (LFD).

In some embodiments the light absorbing material may be a particle, for example a latex particle or a metallic particle such as a gold particle. In LFD embodiments such particles may be conjugated to an antibody and may become concentrated in the test region in response to the presence of analyte leading to light scattering or other light quenching effects that are herein termed “absorption”.

In certain embodiments the OLED of the optical detection unit may comprise as the light emitting component a phosphorescent iridium complex, for example Ir(ppy)₃. In other embodiments the OLED may comprise a light emitting polymer whereby emission is fluorescent in character.

In certain embodiments the optical detection unit may comprise a light absorbing polymer donor and a fullerene acceptor. Preferably the polymer donor comprises a regioregular polythiophene. In the optical detection unit absorption of light by the polymer donor and subsequent transfer of an electron to the acceptor is detected as a photocurrent at the electrodes.

The disclosure further provides an assay device for the quantitative determination of the concentration of at least one analyte in a liquid sample. The device comprises a planar emitter having an emission spectrum E within the wavelength range from λ₁ to λ₂; a planar detector having a light detection spectrum S within the wavelength range from λ₁ to λ₂; and a lateral flow membrane interposed between the emitter and the detector. The device further comprises a conjugate pad that is in fluid communication with a proximal end of the lateral flow membrane, and the conjugate pad comprises optically detectable tagging particles that are bound to a first assay component and have an absorbance spectrum A within the wavelength range from λ₁ to λ₂. A wicking pad is in fluid communication with a distal end of the lateral flow membrane. The lateral flow membrane is formed from a light transmissive material and is capable of transporting fluid from the conjugate pad to the wicking pad by capillary action. The lateral flow membrane comprises at least one test region comprising an immobilised second assay component for retaining the tagging particles in the test region in dependence on the binding between the analyte, the first assay component and the second assay component in order to generate a concentration of tagging particles in the test region that is indicative of the concentration of the analyte in the liquid sample. The emitter comprises an emission layer of an organic electroluminescent material and the emission layer is aligned with the test region of the lateral flow membrane, so that the emitter can illuminate the test region. The detector comprises an absorption layer of an organic photovoltaic material and the absorption layer is aligned with the test region of the lateral flow membrane so that the detector can detect light from the test region. M defines a relationship between E, S and A, and M is less than about 0.4:

$$M = \frac{{\int }_{λ1}^{λ2} {10}^{- A} . E . S d λ}{{\int }_{λ1}^{λ2} E . S d λ} .$$

This embodiment thus provides a device that may be a lateral flow device in which the spectra of the organic emitter, the organic detector, and the optically detectable tagging particles are matched to each other in a manner that provides improved detection of the tagging particles. The value of M is less than about 0.4. Preferably, the value of M is less than about 0.3, more preferably the value of M is less than about 0.2, and most preferably the value of M is less than about 0.1. These values of M permit absorbance measurements to be made using the unit with low background signal, reduced noise, and thereby enable more sensitive and accurate absorbance measurements.

In certain embodiments of the device the required M value can be met without the need for an optical filter such as a narrow bandpass filter that may otherwise be required for example if using inorganic emitters or detectors.

In some embodiments of the device the light absorbing material may be a particle, for example a latex particle or a metallic particle such as a gold particle.

In certain embodiments of the device the OLED of the optical detection unit may comprise as the light emitting component a phosphorescent iridium complex, for example Ir(ppy)₃. In other embodiments the OLED may comprise a light emitting polymer whereby emission is fluorescent in character.

In certain embodiments of the device the optical detection unit may comprise a light absorbing polymer donor and a fullerene acceptor. Preferably the polymer donor comprises a regioregular polythiophene.

### BRIEF DESCRIPTION OF THE DRAWINGS

Embodiments of the invention are further described hereinafter with reference to the accompanying drawings, in which:

FIG. 1A is an illustration of an assay device comprising an optical detection unit according to an embodiment of the present invention;

FIG. 1B is an illustration of a further view of an assay device comprising an optical detection unit according to the embodiment of FIG. 1A;

FIG. 2 is an illustration of an optical detection unit according to a further embodiment of the present invention;

FIG. 3 is an illustration of a component of an embodiment of an assay device according to the present invention;

FIG. 4 is an illustration of a 1-row pixel pattern of an embodiment of an assay device according to the present invention;

FIG. 5 is an illustration of a 2-row pixel pattern of an embodiment of an assay device according to the present invention;

FIG. 6 is an illustration of a 3-row pixel pattern of an embodiment of an assay device according to the present invention;

FIG. 7 is an illustration of a 4-row pixel pattern of an embodiment of an assay device according to the present invention;

FIGS. 8a and 8b show the dose response curves of Kappa and Lambda FLC assays according to Example 1;

FIG. 9 shows the dose response curves of an opiate assay according to Example 2;

FIG. 10 shows the emission, absorption and detection spectra of the device of an optical detection unit according to an embodiment of the present invention and as described in Example 3;

FIG. 11 shows the detection spectra of a series of organic photodiodes with different active layer thicknesses for use in units according to an embodiment of the present invention.

### DETAILED DESCRIPTION

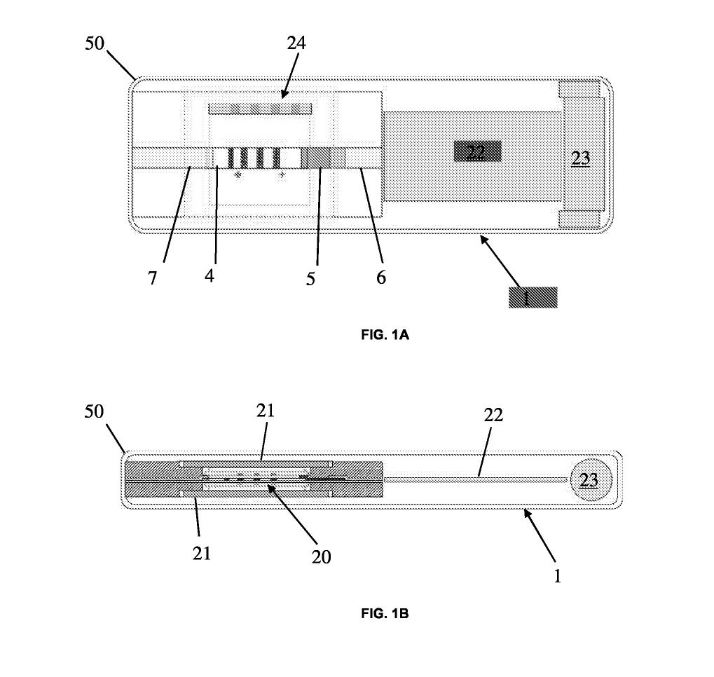

As shown in FIG. 1A and FIG. 1B, according to one embodiment of the present invention, there is provided an assay device 1 contained in a thin, substantially cuboidal housing 50. FIG. 1B provides a side-on illustration of the schematic diagram for the same device as illustrated in FIG. 1A. One end of the housing contains a testing module 20 provided in the plane of the length and width of the housing 50. The opposite end of the housing 50 accommodates a cylindrical battery 23 flat against the wall of the housing 50. Between the testing module 20 and the battery 23 is a printed circuit board 22 which extends from the battery into the length of the housing in the same plane as the testing module 20. Electronics in the testing module 20 are connected to the printed circuit board 22 via an electrical interface 24. The testing module 20 contains a sample pad 6, in fluid communication with a conjugate pad 5. The present conjugate pad 5 contains particle tags which are capable of binding to an assay component. A lateral flow membrane 4 is connected between the conjugate pad 5 and a wicking pad 7. A support structure 21 secures the testing module 20 in the housing 50.

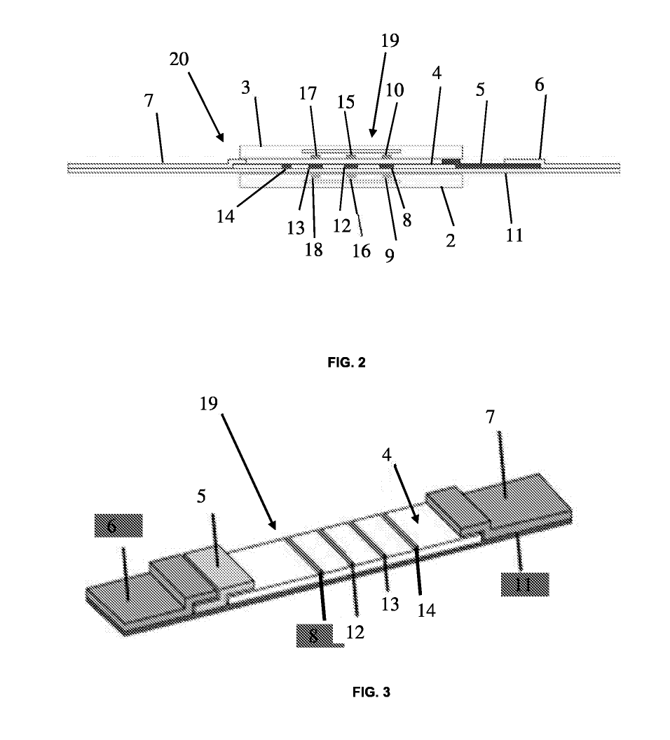

FIG. 2 illustrates an optical detection unit 20 according to an embodiment of the present invention. When a sample is deposited on the sample pad 6, a reservoir of excess sample is formed. The excess sample migrates to the conjugate pad 5. This migration is first caused by the conjugate pad 5, then the wicking action of the lateral flow membrane 4 and then additionally the wicking pad 7. The lateral flow membrane 4 is formed from nitrocellulose. The conjugate pad 5 contains analyte tags. The analyte tags bind to the corresponding available analyte. Capillary action causes the liquid sample, containing any tagged analytes, to flow down the lateral flow membrane 4 from the conjugate pad 5 into the testing area 19 towards the wicking pad 7. Before the sample reaches the wicking pad 7, it encounters a reaction line 8 containing fixed receptors for the analyte. When the tagged analyte reaches this point, the receptors bind to the analyte, holding the analyte and the tags in place. The presence of the coloured analyte tag will cause the reaction line 8 to change colour as the concentration of the tags increases. In the presently described example, the concentration of the coloured tags is a direct indicator of the concentration of analyte at the reaction line which provides an indication of the concentration of the analyte in the liquid sample.

The above is an example of a sandwich assay technique. A competitive assay is also possible in which the intensity of the response from the reaction line 12 (usually a colour) is inversely proportional to the amount of analyte present in the sample. In one example of this technique, the conjugate pad 5 additionally contains a pre-tagged second analyte or analyte analogue. The analyte from the sample passes unchanged through the conjugate pad 5, and will bind to the receptors on a further reaction line 12, occupying receptor sites to which the pre-tagged analytes or analyte analogues would otherwise bind. The less analyte there is in the sample, the more pre-tagged analyte or analyte analogue is able to bind to the receptors, resulting in a stronger colouring of the line. In a further example of this technique, the conjugate pad 5 could also or instead contain a tagged receptor. In this case fixed analyte or analyte analogue is immobilised on a reaction line. The more analyte present in the sample, the more of the tagged receptor that will bind to the analyte from the sample, and so not be available to bind to the fixed analyte or analyte analogue. The competitive assay technique may be used to qualitatively test for the absence of a particular analyte, though is not a purely binary test, and a very small amount of analyte in the sample is still likely to result in binding of the pre-tagged molecule (be that analyte, analyte analogue or receptor) at the position of the line. The competitive assay technique may instead be used to quantitatively indicate the concentration of a particular analyte in the liquid sample.

There is also a further line 13 of control receptors on the lateral flow membrane 4 which react with the tagged component itself. The control line 13 contains immobilised receptors which bind to the tagged component. The control line 13 should become coloured whenever the test is carried out, regardless of whether the sample contains any analyte. This helps confirm the test is performing correctly. In the presently described example, the reaction line 8 only changes colour when the analyte is present in the sample. In embodiments with multiple assays, there may be multiple control lines. In this way, the control lines can be used to determine whether each test to be performed by the lateral flow device has been performed. The control line 13 in the current example is provided downstream of the earlier reaction lines. By providing the control line 13 downstream of the reaction lines, the analyte tag must flow through the other reaction lines before they can bind to the control line indicating that a test has been carried out.

In the present case, the lateral flow membrane 4 is approximately 100 μm thick and the reaction lines 8, 12 and control line 13 are each 1.0 mm×5.0 mm with a 2.0 mm gap between them. The lateral flow membrane is formed from nitrocellulose. The sample pad 6, conjugate pad 5, lateral flow membrane 4 and wicking pad 7 are provided on a transparent substrate 11.

A reference line 14 is provided on the lateral flow membrane 4 and is used for alignment during construction of the testing area 19. The reference line 14 is typically thinner than the reaction lines 8, 12 or control line 13. The reference line in the current example is 0.5 mm×5.0 mm with a 1.5 mm gap between the control line 13.

Whilst the examples disclose analysing the presence, absence, or concentration of a range of analytes in the sample, it is possible to perform this analysis with fewer or more analyte tests. A range of different tags and receptor lines can be used to determine the presence, absence, or concentration of multiple different analytes. The presence of some analytes may be tested in combination with the absence of different, or the same, analytes. Tests for example assays are given in Table 1 below. In each case, the purpose of the test is given, along with the first assay component, second assay component, the analyte of interest, and which type of assay (sandwich or competitive). All assays can be performed using analyte or antibodies to the analyte labelled with any type of labelling particle. Example labelling particles include gold nano-particles, coloured latex particles, or fluorescent labels. As can be readily identified from the table in row N, assays for other analytes can be constructed using analyte antigens as the first component and antibodies to the analyte as the second component where the assay type is sandwich. Where the assay type is competitive (row M), the antibodies to the analyte would be the first component, and the analyte antigen would be the second component.

TABLE 1

| | | | Label Binder | Immobilised | | Assay Type |
|---|---|---|---|---|---|---|
| | | | (first | Line (second | | (Sandwich/ |
| | Test for: | Label | component) | component) | Analyte | Competitive) |
| A | Myeloma | All | Antibodies to | Kappa FLC | Kappa FLC | Competitive |
| | | | free kappa | antigen | | |
| | | | light chains | | | |
| | | | (k-FLC) | | | |
| B | Myeloma | All | Antibodies to | Lambda FLC | Lambda FLC | Competitive |
| | | | free lambda | antigen | | |
| | | | light chains | | | |
| | | | (l-FLC) | | | |
| C | Myeloma | All | Antibodies to | Antibodies to | Kappa FLC | Sandwich |
| | | | free kappa | free kappa light | | |
| | | | light chains | chains (k-FLC) | | |
| | | | (k-FLC) | | | |
| D | Myeloma | All | Antibodies to | Antibodies to | Lambda FLC | Sandwich |
| | | | free lambda | free lambda | | |
| | | | light chains | light chains | | |
| | | | (l-FLC) | (l-FLC) | | |
| E | Opiates | All | Antibodies to | Opiates | Opiates | Competitive |
| | | | Opiates | antigen | | |
| F | Amphetamines | All | Antibodies to | Amphetamines | Amphetamines | Competitive |
| | | | Amphetamines | antigen | | |
| G | Benzodiazepines | All | Antibodies to | Benzodiazepines | Benzodiazepines | Competitive |
| | | | Benzodiazepines | antigen | | |
| H | Cannabis | All | Antibodies to | Cannabinoid | Cannabis | Competitive |
| | | | Cannabinoids | derivative | | |
| | | | | antigen | | |
| I | Cocaine | All | Antibodies to | Cocainoids | Cocaine | Competitive |
| | | | Cocainoids | antigen | | |
| J | Methamphetamine | All | Antibodies to | Methamphetamine | Methamphetamine | Competitive |
| | | | Methamphetamine | antigen | | |
| K | Methadone | All | Antibodies to | Methadone | Methadone | Competitive |
| | | | Methadone | antigen | | |
| L | Phencyclidine | All | Antibodies to | Phencyclidine | Phencyclidine | Competitive |
| | (PCP) | | Phencyclidine | (PCP) antigen | (PCP) | |
| | | | (PCP) | | | |
| M | Others | All | Antibodies to | Others | Others | Competitive |
| | | | Others | antigen | | |
| N | Others | All | Antibodies to | Antibodies to | Others | Sandwich |
| | | | Others | Others | | |
| O | Troponin I | All | Antibodies to | Antibodies to | Troponin I | Sandwich |
| | | | Troponin I | Troponin I | | |
| P | Myoglobin | All | Antibodies to | Antibodies to | Myoglobin | Sandwich |
| | | | Myoglobin | Myoglobin | | |
| Q | CKMB | All | Antibodies to | Antibodies to | CKMB | Sandwich |
| | | | CKMB | CKMB | | |
| R | Cortisol in saliva, | All | Antibodies to | Cortisol | Cortisol | Competitive |
| | serum or urine | | Cortisol | antigen | | |

Whilst common household assay tests, such as some pregnancy tests, have an apparently binary result and require a user to manually interpret the results, the present device uses an Organic Light Emitting Diode (OLED) and opposed Organic Photo Diode (OPD) to measure the light absorption as a result of the analyte test. Whilst the presently described embodiment uses the absorption of light by a substance to indicate the concentration of an analyte in a test sample, embodiments can equally be envisaged where the tag on the analyte is luminescent and emits light itself, either as a result of fluorescence, phosphorescence, or as a result of a chemical or electrochemical reaction.

The assays for Myeloma are described in rows labelled A-D in Table 1. To test for myeloma, the ratio of Kappa FLC concentration to Lambda FLC concentration is determined.

The OLED illuminates the sample with light having known characteristics (intensity, wavelength, etc). When light is received by the OPD, a current is generated. By measuring this current, the light absorbed by the immobilised labels at the reaction line, 8, 12 and surrounding membrane can be determined. This gives an indication of the concentration of tagged analyte present in the sample.

The OLED is a layered structure sitting on a plastic substrate (PET), a glass substrate, or an laminate comprising plastic layers alternating with inorganic barrier layers. The OLED is formed from a layer of patterned ITO (indium tin oxide, which is conductive and transparent), a layer of hole injection material, a layer of active material, and a cathode. It is possible to maximize the forward emission of the device by tuning the thicknesses of the ITO and more importantly the active material and cathode. With such modifications in the stack geometry the amount of light being emitted perpendicular to the device can be maximised. This will mean that a larger proportion of light emitted by the OLED passes through the membrane, and impinges onto the OPD. Conventional inorganic LEDs with epoxy protection have a Lambertian emission, and therefore waste a significant amount of light.

In the present example, the OLED 2 contains emission regions 9, 16, 18, provided opposite the organic photovoltaic cell (OPD) 3, containing detection regions 10, 15, 17. The emission light colour of all three regions in the present example is blue, as they are formed from a layer of the same material. Similarly, in the present example, the material of the OPD regions 10, 15, 17 is optimised to detect blue light.

The OLED emission regions 9, 16, 18 and OPD detection regions 10, 15, 17 are sized to sit within the footprint of the reaction lines 8, 13, 14 containing bound receptors set up to catch and bind the tagged analyte (be that pre-tagged or otherwise). In the present case, this results in pixels 0.9 mm×4.9 mm. This maximises the proportion of the light emission from the OLED that is capable of interacting with the tagged analyte and the surrounding lateral flow membrane 4. Another factor which improves the proportion of the emitted light that can interact with the membrane and tagged analyte is the proximity of both the OLED and the OPD to the lateral flow membrane 4. In the present example, only the barrier material is interposed between the OLED/OPD and the membrane, with a thickness of approximately 100 μm.

The circuit board 22 and battery 23 included within the housing 50 for the assay device 1 control and power the OLED and OPD. The circuit board 22 also includes a microprocessor suitable for performing basic analysis in order to calculate a quantitative value representative of the amount of the analyte(s) present in the sample and/or ratios thereof.

For an example OPD the following structure can be used. The first layer (closest to the membrane) is a pre-patterned indium-tin-oxide (ITO) glass substrate. The glass substrate provides a barrier layer for the OPD. On top of the ITO layer is provided a 50 nm thick layer of Baytron P grade poly(styrenesulphonate)-doped poly(3,4-ethylenedioxythiophene) (PEDOT:PSS) and 10 nm thick Poly(methyl methacrylate) (PMMA)film interlayer is provided thereon. The active layer is 165 nm thick regioregular poly(3-hexylthiophene):1-(3-Methoxycarbonylpropyl)-1-phenyl-[6.6]C61 (P3HT:PCBM) with an upper electrode for the device of 100 nm-thick aluminium.

This is only one example of an OPD suitable for use in embodiments of the present invention. The skilled person will be aware of methods of manufacturing such OPDs and other materials from which suitable OPDs may be manufactured.

The OPD active layer typically comprises a donor and an acceptor, which may be selected from among those known in the art for polymer solar cells (see, e.g. Li, G., Zhu, R. and Yang Y. (2012) Nature Photonics 6:153-161). A donor material may be selected according to its absorbance in the wavelength range relevant to the quencher to be used in the assay. However, other factors than material selection can affect the detection spectrum of an OPD. These factors include the morphology of the acceptor and donor heterostructure, which can be influenced by the solvent and drying conditions used to prepare the device; the thickness of the active layer; the materials used in adjacent layers such as charge transporting layer; the electrodes used; microcavity effects arising from the combination of layer thicknesses and refractive indices of the layer materials; and light incoupling structures such as distributed Bragg reflectors. The skilled person is therefore aware of a number of structural and material factors that may be used to better tailor the detection spectrum of the OPD to the assay for which it is needed.

Likewise, the skilled person is aware of several structures and material combinations from which to fabricate OLEDs suitable for the present invention. In one particular OLED type, the structure is a plastic substrate (PET), a layer of patterned ITO, a layer of hole injection material, a layer of active material, and a cathode. In particular, the emission spectrum of the OLED can be tailored by the choice of the organic polymer or other small molecule. For example, iridium containing complexes typically have well-defined phosphorescent emission spectra and the peak wavelength can be varied across the visible spectrum by changing the ligands to which the metal is bound. For example, and without limitation, these complexes and their peak emissions can include fac-Ir(ppy)₃ (519 nm), fac-Ir(4′,6′-dfppy)₃ (467 nm), fac-Ir(atpy)₃ (581 nm), (piq)₂Ir(acac) (622 nm), (niq)₂Ir(acac), fac-Ir(pmi)₃ (380 nm), and solubilized derivatives or dendrimeric derivatives thereof. In addition to the selection of emissive materials, other features of the OLED may be used to tailor the emission spectrum to a particular application. These features include the materials used as the host for the emissive layer, or materials in adjacent layers such as hole or electron transporting layers; the electrodes used; microcavity effects arising from the combination of layer thicknesses and refractive indices of the layer materials; the drive voltage applied to the OLED; light outcoupling structures such as distributed Bragg reflectors. The skilled person is therefore aware of a number of structural and material factors that may be used to better tailor the emission spectrum of the OLED to the assay for which it is needed.

The skilled person is also aware of a wide range of choices available in the biological assay field for the selection of a light quencher (the coloured tags used to label the compound of interest) that may, for example, be conjugated to an antibody. Gold nanoparticles can be used and in this case, a green illumination source should be used. Alternatively, blue polystyrene labels can be used and this case, a red illumination source should be used. In addition, a wide variety of organic quenchers are available as, for example, the dabcyl, QSY®, and DyLight™ quencher families obtainable from ThermoFisher.

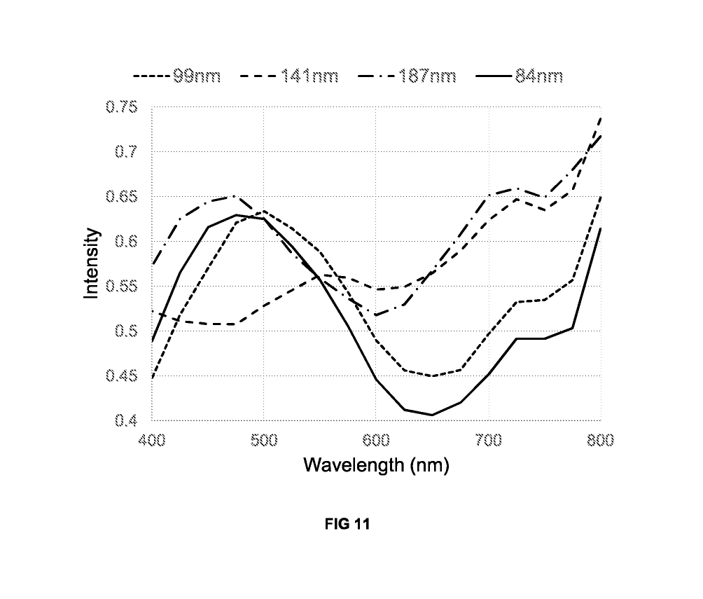

To optimize the specificity and sensitivity of an absorbance assay, the emission spectrum E of the OLED, the light detection spectrum S, and the absorbance spectrum A of the light absorbing component must be correctly matched. A mismatch between the absorbance spectrum and the emission spectrum will result in an undesireable background signal unless the detection spectrum is tailored to have low sensitivity at wavelengths that are emitted but not absorbed. Similarly, if the detection spectrum has low values at wavelengths where emission is strong but absorption is weak, the sensitivity of the assay will be reduced. This three-way matching is non-trivial particularly where subtle changes in spectra (e.g. see FIG. 11) require objective means to ascertain which combination of OLED, OPD and quencher may provide optimum detection.

The present inventors have found that the following relation provides optimum matching between the emission spectrum E of the OLED, the light detection spectrum S, and the absorbance spectrum A of the light absorbing component for an absorbance-based assay:

$$M = \frac{{\int }_{λ1}^{λ2} {10}^{- A} . E . S d λ}{{\int }_{λ1}^{λ2} E . S d λ}$$

There are a large number of ways in which the matching between E, S and A may be expressed mathematically, and the present relation has particularly advantageous features. The relation is selected such that E·S appears in both the denominator and numerator, which has the benefit that the units of E and S are not relevant and can be any suitable units, or normalized to any value, as long as the same method is applied across any group of optical detection units being evaluated. Spectrum A may be measured using a particular test region in its highest absorbing state that is obtained in use by, for example, using a spectrometer or other means for determining the logarithm of the incident to transmitted light as a function of wavelength between λ₁ and λ₂. In use, λ₁ and λ₂ are selected to include between these limiting wavelengths at least the main spectral features of E, S and A.

Low values of M represent a good match between E, S and A as is required for good sensitivity and low background signal in an optical detection unit or in an assay device comprising such an optical detection unit. Thus, in an optical detection unit comprising an OLED and an OPD, M is less than about 0.4. Preferably M is less than about 0.3, more preferably less than about 0.2, and most preferably less than about 0.1.

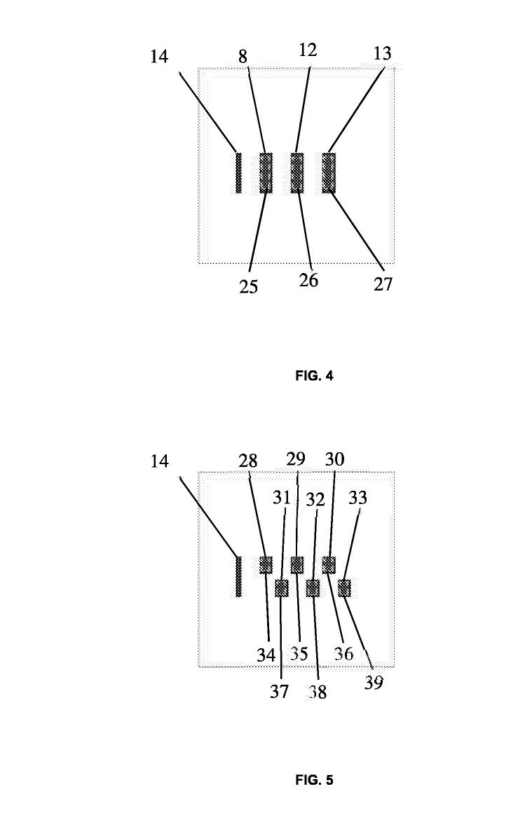

FIG. 4 illustrates a 1-row pixel pattern of an embodiment of an assay device according to the present invention. The reference line 14, reaction lines 8 and 12, and control line 13 are provided on the lateral flow membrane. The OLED and OPD production processes allow pixels of any size and positioning to be created to overlay the reaction and control lines. In FIG. 4, the pixel outlines 25, 26, and 27 shown as dashed lines represent the outline of the OPD sensitive regions and OLED pixels. These pixels are centered on the reaction lines 8, 12 (or control line 13). The pixel outlines 25, 26, and 27 are also smaller than the reaction lines 8, 12 (or control line 13). In this way, the light which enters the OPD from the OLED without passing through the reaction line (i.e. passing through a part of the lateral flow membrane not forming part of the reaction line or control line) is minimized and/or substantially eliminated. In some embodiments, the pixel outlines may have substantially the same extent as the reaction lines. The reaction lines 8, 12 may be correspond to assays for the same analyte. In this way, the accuracy of any resulting indications of the analyte concentration in the liquid sample can be maximised by multiple assays of the same sample.

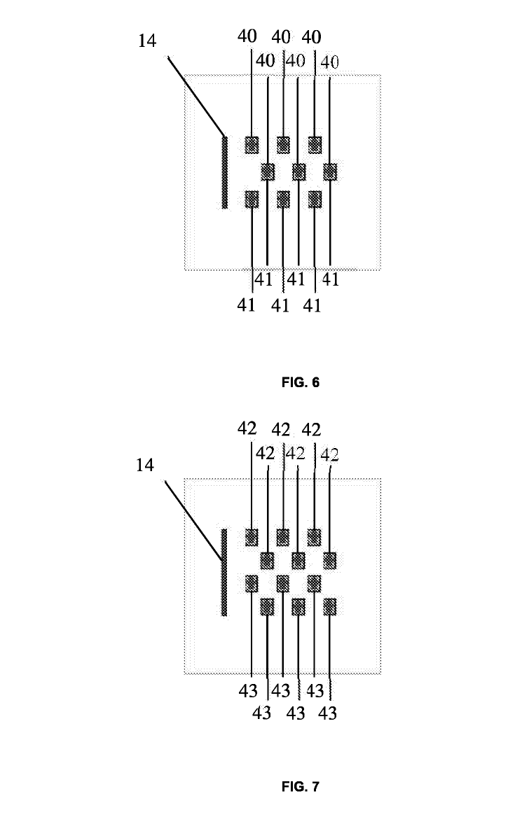

FIG. 5 illustrates a 2-row pixel pattern of an embodiment of an assay device according to the present invention. In this embodiment, there are two parallel lateral flow membranes. As described previously, the reference line 14 is used to align the reaction regions 28, 29, 30, 31, 32, 33 with the OPD and OLED outlines 34, 35, 36, 37, 38, 39 respectively. By diagonally offsetting the matched reaction regions (lines) from each other, the light bleed between two neighbouring reaction regions, is minimised. In this way, for example, the amount of light from the OPD/OLED outline 37 detectable by the OPD on the OPD/OLED outline 34, 35 is minimised. This allows a particularly compact arrangement of assays in a single assay device. In some embodiments, each parallel lateral flow membrane can contain a single reaction region, with each lateral flow membrane testing for a different analyte. In other embodiments, each parallel lateral flow membrane can contain a single or multiple reaction regions, with each lateral flow membrane testing for the same one or group of analytes. This allows the accuracy of the resulting indications of the analyte concentrations in the liquid sample to be improved. In yet other embodiments, multiple testing regions on a plurality of parallel lateral flow membranes can be used to test for the same analyte in different ways. In this way, one lateral flow membrane may test for a given analyte using a sandwich assay technique, whilst another lateral flow membrane may test for the same given analyte using a competitive assay technique.

FIGS. 6 and 7 illustrate respectively a 3-row and 4-row pixel pattern of an embodiment of an assay device according to the present invention. The reaction regions 40, 42 provided on the lateral flow membrane are arranged to minimise light from the OLED having outline 41, 43 bleeding into the outline of any neighbouring OPD having outline 41, 43. As before, the reference line 14 is provided for alignment purposes.

Whilst in the embodiments shown, the reaction lines and/or reaction regions are intended to extend to each side of each lateral flow membrane, as seen specifically in reaction line 12 from FIG. 3, the invention extends to alternative embodiments where the reaction lines and/or reaction regions do not extend to each side of each lateral flow membrane. For example, the reaction regions may be centred in the middle of the lateral flow membrane. Alternatively, two distinct regions may be provided side-by-side on a lateral flow membrane. There may be a space on the lateral flow membrane between the two reaction regions. In some embodiments, the two reaction regions are provided in contact with each other. In some embodiments, two or more regions may be spaced or offset both in the proximal-distal direction, and in the width direction of the lateral flow membrane. The reaction regions may be provided on distinct lateral flow membranes which may be provided, for example, side-by-side.

Whilst embodiments of the present invention have been described using direct tagging, indirect tagging is also possible. In embodiments where a first antibody binds to the analyte, the tagging particle may be bound to a further antibody, which is configured to bind to the first antibody. In this way the same labelled antibody can be used for several different analytes.

Whilst the embodiments shown use a conjugate pad, it will be appreciated that the sample may be pre-treated with the analyte tags. This may ensure better mixing and binding between the analyte and analyte tags, particularly where there are very low concentrations of analyte. In this case, the conjugate pad is not required, and the pre-treated sample may be deposited on the sample pad or the lateral flow membrane directly. In some embodiments where the presence or concentration of multiple analytes is to be tested, the sample may be pre-treated for only some of the analytes of interest. In this case, a conjugate pad is still required.

Whilst the embodiments shown are for quantitative measurements, it will be appreciated that the invention is equally applicable to qualitative or semi-quantitative assay devices, where only a presence or absence indication of one or more analytes of interest is required. In semi-quantitative assay devices, only a discretised reading of, for example, a plurality concentration levels is required. The concentration levels need not be regularly spaced over the range of concentration to be measured.

An advantage of the present invention in embodiments using fabricated OPDs and OLEDs compared to prior art devices using silicon-based inorganic detectors or GaAs and/or InGaAs and/or SbGaInAs-based inorganic emitters is the ability to provide multiple assays (quantitative or otherwise) without a corresponding increase in material costs. In the inorganic emitters and detectors of the prior art, multiple reaction regions require multiple emitters and detectors, which each have a unit cost. In embodiments of the present invention, OPDs and OLED are fabricated from a single piece, regardless of the number of pixels the emitter or detector requires, and so there is only a minimal increase in cost for the provision of an additional reaction region.

### Example 1

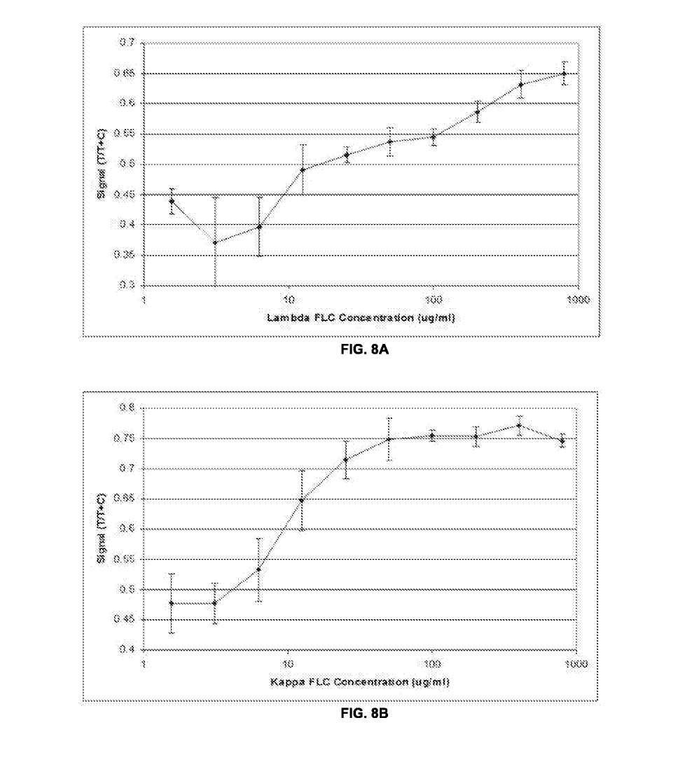

An organic light emitting diode (OLED) has three pixels in the manner of the embodiment of FIG. 4 and emits green light with a wavelength of 520 nm and an organic photodiode (OPD) has the same pattern as the OLED. The lateral flow membrane comprises one control region and two test regions. The first assay is Kappa FLC antigen and the second assay is Lambda FLC antigen. When an amount of a sample containing Kappa and Lambda FLC antigen flows along the membrane, tagged antibodies combine with Kappa and Lambda FLC antigens in the sample or on the membrane. More antigens in the sample generate less colour and more light is transmitted through the membrane so that a larger signal is detected by the OPD. FIG. 8 shows the dose response curves of the Kappa and Lambda FLC assays.

### Example 2

An organic light emitting diode (OLED) has a configuration as shown in FIG. 5 but only two of three pixels are operated in each row. The emitting wavelength is 520 nm. The organic photo diode (OPD) has the same pattern as the OLED. The lateral flow membrane comprises one control region and one test region of opiates antibody. Two identical lateral flow membrane stripes are aligned in parallel with two rows of OLED and OPD pairs to improve the accuracy by running samples twice simultaneously. When a sample including a certain amount of opiates antigen flows along the membrane, the antigen combines with tagging material (gold beads) and binds with opiates antibody on the membrane. More antigens in the sample generate darker colour and less light transmits through the membrane so that weaker signal is detected by the OPD. FIG. 9 is a dose response curve for the opiates assay.

### Example 3

A device was prepared substantially as shown in FIGS. 1 and 2 in which the OLEDs were manufactured by solution processing and had the following structure:

glass/ITO/polymer hole transport layer/polymer host, Ir-dendrimer phosphorescent green emitter/Ag

The OPDs of the present example were also manufactured by solution processing and had the following structure: glass/ITO/polymer hole transport layer/polymer donor and acceptor/Ag

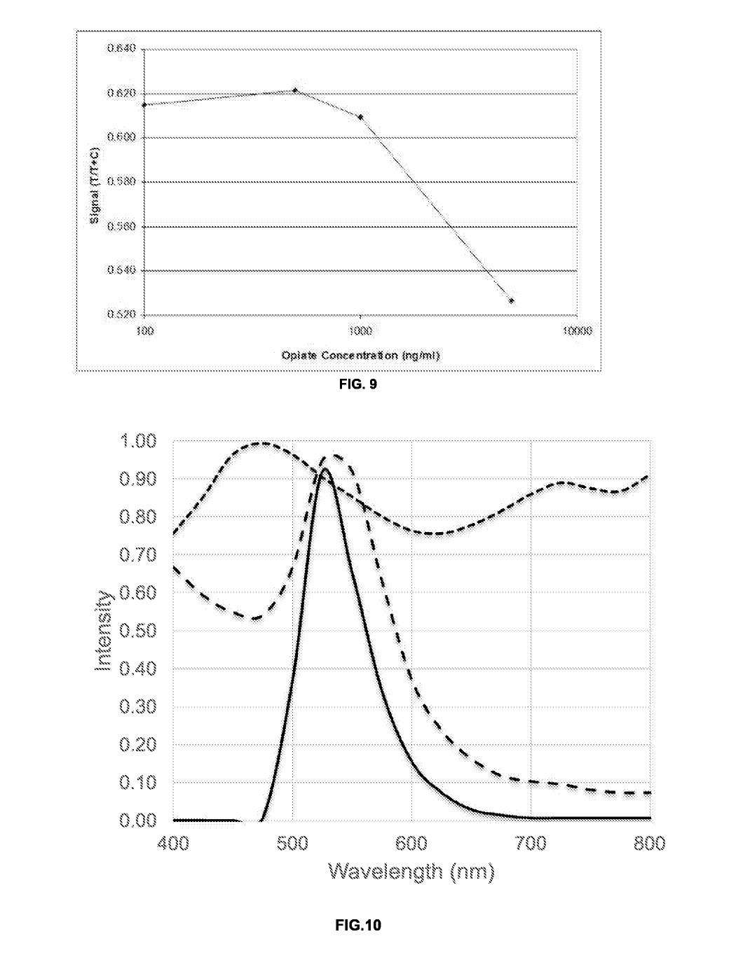

The device of this example can be used for assays of the kind described in Examples 1 and 2 in which the light absorbing component is a gold bead. FIG. 10 shows the emission, absorption and detection spectra of the optical detection unit of this example. The value of M for this example was 0.19.

### Example 4

TABLE 2

| Thickness (nm) | M | Ranking |
|---|---|---|
| 84 | 0.217 | 1 |
| 99 | 0.220 | 2 |
| 141 | 0.238 | 4 |
| 187 | 0.235 | 3 |

A series of OPDs in the manner of those according to Example 3 was prepared in which the thickness of the solution deposited active layer comprising the donor and acceptor was varied as shown in Table 2 to include active layer thicknesses of 84 nm, 99 nm, 141 nm and 187 nm. Changes in the thickness of the active layer altered the detection spectrum of the OPDs in a complex manner as shown in FIG. 11 in which intensity denotes photocurrent. The complex spectral changes mean that selection of the optimal OPD for a particular application may not be readily apparent from visual inspection of the spectra alone. Ranking the OPDs of this example for use in an optical detection unit, for example one in which these OPDs are combined with an OLED emitter and gold particle quenchers, the spectra of which are shown in FIG. 10, requires objective assessment. This problem is solved using the M value. Table 2 shows the thicknesses of the active layers, their M value when used in combination with the OLED emitter and gold particle absorbance of FIG. 10, and their resulting performance. The present disclosure thus provides an optical detection unit for an assay device for the quantitative determination of the concentration of an analyte in a liquid sample, and an assay device comprising the same. The detection unit comprises an organic light emitting diode (OLED) emitter that has an emission spectrum E within the wavelength range from λ₁ to λ₂, and an organic photodiode detector (OPD) that has a light detection spectrum S within the wavelength range from λ₁ to λ₂. The detection unit has a test region that comprises a light absorbing component that has an absorbance spectrum A within the wavelength range from λ₁ to λ₂. The test region is positioned adjacent to the emitter and the detector to form an optical pathway from the light emitting diode to the photodiode through at least a portion of the test region. Formula M defines a relationship between E, S and A, and M is less than about 0.4.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An optical detection unit for an assay device for the quantitative determination of the concentration of an analyte in a liquid sample, the detection unit comprising:
an organic light emitting diode (OLED) emitter having an emission spectrum E within the wavelength range from λ₁ to λ₂;
an organic photodiode detector (OPD) having a light detection spectrum S within the wavelength range from λ₁ to λ₂; and
a test region, the test region comprising a light absorbing component having an absorbance spectrum A within the wavelength range from λ₁ to λ₂;
wherein the test region positioned adjacent to the emitter and the detector to form an optical pathway from the light emitting diode to the photodiode through at least a portion of the test region; and
wherein formula M defines a relationship between E, S and A, and M is less than about 0.4:
$$M = \frac{{\int }_{λ1}^{λ2} {10}^{- A} . E . S d λ}{{\int }_{λ1}^{λ2} E . S d λ}$$

2. The optical detection unit of claim 1, wherein the optical pathway does not comprise an optical filter.

3. The optical detection unit of claim 1, wherein the test region comprises a light transmissive lateral flow membrane

4. The optical detection unit according to claim 1, wherein M is less than about 0.3.

5. The optical detection unit of claim 4, wherein M is less than about 0.2.

6. The optical detection unit of claim 5, wherein M is less than about 0.1.

7. The optical detection unit as claimed in claim 1, wherein the light absorbing component is a metallic or latex particle.

8. The optical detection unit of claim 7, wherein the light absorbing component is a gold particle.

9. The optical detection unit as claimed in claim 1, wherein the OLED comprises a phosphorescent iridium complex.

10. The optical detection unit as claimed in claim 1, wherein the OPD comprises a light absorbing polymer donor and a fullerene acceptor.

11. The optical detection unit according to claim 10, wherein the polymer donor comprises regioregular polythiophene.

12. An assay device for the quantitative determination of the concentration of at least one analyte in a liquid sample, the device comprising:
a planar emitter having an emission spectrum E within the wavelength range from λ₁ to λ₂;
a planar detector having a light detection spectrum S within the wavelength range from λ₁ to λ₂;
a lateral flow membrane interposed between the emitter and the detector;
a conjugate pad in fluid communication with a proximal end of the lateral flow membrane, the conjugate pad comprising optically detectable tagging particles bound to a first assay component and having an absorbance spectrum A within the wavelength range from λ₁ to λ₂; and
a wicking pad in fluid communication with a distal end of the lateral flow membrane,
wherein the lateral flow membrane is formed from a light transmissive material and is capable of transporting fluid from the conjugate pad to the wicking pad by capillary action,
wherein the lateral flow membrane comprises at least one test region comprising an immobilised second assay component for retaining the tagging particles in the test region in dependence on the binding between the analyte, the first assay component and the second assay component in order to generate a concentration of tagging particles in the test region that is indicative of the concentration of the analyte in the liquid sample,
wherein the emitter comprises an emission layer of an organic electroluminescent material and the emission layer is aligned with the test region of the lateral flow membrane, whereby the emitter is capable of illuminating the test region,
wherein the detector comprises an absorption layer of an organic photovoltaic material and the absorption layer is aligned with the test region of the lateral flow membrane,
whereby the detector is capable of detecting light from the test region, and
wherein formula M defines a relationship between E, S and A, and M is less than about 0.4:
$$M = \frac{{\int }_{λ1}^{λ2} {10}^{- A} . E . S d λ}{{\int }_{λ1}^{λ2} E . S d λ} .$$

13. The assay device as claimed in claim 12, wherein the optical pathway does not comprise an optical filter.

14. The assay device as claimed in claim 12, wherein M is less than about 0.3.

15. The assay device as claimed in claim 14, wherein M is less than about 0.2.

16. (canceled)

17. The assay device as claimed in claim 12, wherein the light absorbing component is a metallic or latex particle.

18. The assay device as claimed in claim 17, wherein the light absorbing component is a gold particle.

19. The assay device as claimed in claim 12, wherein the OLED comprises a phosphorescent iridium complex.

20. The assay device as claimed in claim 12, wherein the OPD comprises a light absorbing polymer donor and a fullerene acceptor.

21. The assay device as claimed in claim 20, wherein the polymer donor comprises regioregular polythiophene.
